(19) United States
(12) Patent Application Publication
(10) Pub. No.: US 2019/0382726 A1
(43) Pub. Date: Dec. 19, 2019

(54) METHOD, DEVICE AND KIT FOR DETERMINING CARDIAC CONDUCTION

(71) Applicant: Universität Rostock, Rostock (DE)

(72) Inventor: Robert David, Mering (DE)

(21) Appl. No.: 16/464,030

(22) PCT Filed: Nov. 24, 2017
(86) PCT No.: PCT/EP2017/080304

§ 371 Date: May 24, 2019

(30) Foreign Application Priority Data
Nov. 25, 2016 (DE) 10 2016 223 423.4

(51) Int. Cl. C12N 5/077; G01N 33/50; A61N 1/37; A61N 1/362

(52) U.S. Cl. CPC C12N 5/0657; G01N 33/5061; C12N 15/85; A61N 1/3627; A61N 1/3629; A61N 1/3702

(57) ABSTRACT

The invention relates to a method for determining cardiac conduction, especially the cardiac conduction of a test subject, comprising:
(i) providing induced sinoatrial bodies (iSABs) comprising cardiac pacemaker cells;
(ii) providing working-myocardium cardiomyocytes, especially working-myocardium cardiomyocytes of the test subject;
(iii) measuring the cardiac conduction;
   the induced sinoatrial bodies according to (i) and the working-myocardium cardiomyocytes according to (ii) being arranged in a spatially separated manner, but in conductive communication with one another.

The present invention relates to a method for determining cardiac conduction, especially the cardiac conduction of a test subject, comprising providing induced sinoatrial bodies (iSABs) comprising cardiac pacemaker cells; providing working-myocardium cardiomyocytes, especially working-myocardium cardiomyocytes of the test subject; and measuring the cardiac conduction; the induced sinoatrial bodies and the working-myocardium cardiomyocytes being arranged in a spatially separated manner, but in conductive communication with one another.

The conduction system of the heart forwards the electrical signals which regulate the pump activity of the heart. At the same time, the basic rhythm of these impulses is generated by the pacemaker system. Both systems, i.e., pacemaker system and conduction system of the heart, contain specialized cardiac muscle cells.

The pacemaker system comprises the cardiac muscle cells of the sinus node and those of the atrioventricular node (AV node), the sinus node serving as the primary impulse initiator (pacemaker) of the heart and the AV node serving as the secondary pacemaker of the heart. Because of the higher frequency of the sinus node—about 60 to 80 stimuli per minute—the pacemaker activity of the AV node, which itself can generate about 40 to 50 stimuli per minute, takes effect only in the event of a failure of the sinus node. In the case of a normally functioning sinus node, the electrical impulses thereof are forwarded via the AV node. Further conduction takes place via the His-Purkinje system, i.e., electrical impulses are forwarded from the AV node up to the bundle of His, which, owing to its inherent rhythm of about 20 to 30 stimuli per minute, is also referred to as the tertiary pacemaker of the heart. Induced by the pacemaker cells of the sinus node, the stimulus spreads via the AV node and the His-Purkinje system to the working myocardium. Here, AV node and bundle of His determine when and whether action potentials are passed on from atrial tissue (atrium, atrial cells) to ventricular tissue (ventricle, ventricular cells).

The conduction system of the heart is responsible for regular and independent cardiac rhythm. Disturbances in conduction may occur when the signals within the conduction system are no longer forwarded properly. Disturbances in conduction may inter alia be caused by diseases, such as, for example, coronary heart diseases or sick sinus syndrome, and by disturbances in electrolyte housekeeping, but also some medicaments such as, for example, digitalis or certain antiarrhythmics may lead to disturbances in conduction. The diagnosis of a disturbance in conduction is normally made by an ECG examination. What are possible as therapy are the treatment of the underlying disease, the dose-reduction or the discontinuation of medicaments with a triggering effect, the use of a pacemaker and the use of medicaments against heart arrhythmias (antiarrhythmics). ECG-based diagnosis has hitherto been possible only directly on the test subject, this sometimes requiring carrying out a long-term ECG examination, usually requiring the ECG instrument to be worn on the body for 24 hours. Investigating medicaments with respect to their effect on conduction and with respect to their suitability for the treatment of disturbances in conduction has hitherto been possible only directly on the test subject or by means of animal experiments.

One of the objects underlying the present invention is therefore that of providing an improved method for determining cardiac conduction, especially with respect to the avoidance of lengthy ECGs on the test subject or the avoidance of animal experiments.

It was found that, surprisingly, such a method can be provided by providing induced sinoatrial bodies (iSABs) comprising cardiac pacemaker cells in conjunction with working-myocardium cardiomyocytes and using them for measuring cardiac conduction.

The invention therefore provides a method for determining cardiac conduction, especially the cardiac conduction of a test subject, comprising:
(i) providing induced sinoatrial bodies (iSABs) comprising cardiac pacemaker cells;
(ii) providing working-myocardium cardiomyocytes, especially working-myocardium cardiomyocytes of the test subject;
(iii) measuring the cardiac conduction;
the induced sinoatrial bodies according to (i) and the working-myocardium cardiomyocytes according to (ii) being arranged in a spatially separated manner, but in conductive communication with one another.

“Arranged in conductive communication with one another” means that the passing-on of electrical impulses, for example from the induced sinoatrial bodies according to (i) to the working-myocardium cardiomyocytes according to (ii), is possible despite spatial separation, there preferably being a spatially limited, direct contact between at least parts of the iSABs according to (i) and at least parts of the working-myocardium cardiomyocytes according to (ii), via which contact it is further preferably possible for connexin connections to form between the iSABs according to (i) and the working-myocardium cardiomyocytes according to (ii). “Spatially limited, direct contact” means that the spatially separated regions in which the iSABs according to (i) and the working-myocardium cardiomyocytes according to (ii) are situated have a common open contact area or a contact region via which the iSABS and the working-myocardium cardiomyocytes can come into contact. Preferably, the method comprises (i), (ii) and (iii). In a preferred embodiment, the method consists of (i), (ii) and (iii). In the context of the present invention, the naming of steps with Roman numerals generally means that said steps are carried out in successive order from the lowest value to the highest value.

Recently, it was possible to show that, for example with the aid of the combination of overexpression of the transcription factor TBX3 and an Myh6 promoter-based antibiotic selection, it is possible to program murine pluripotent stem cells into hitherto unobserved aggregates of pacemaker cells. With 300-400 beats per minute, said “induced sinoatrial bodies” (iSABs) show for the first time a contraction frequency which approaches that of the mouse heart and, at the same time, corresponds to that of explanted, in vitro cultured murine sinus node cells (Jung, J. J., et al., Programming and isolation of highly pure physiologically and pharmacologically functional sinus-nodal bodies from pluripotent stem cells. Stem Cell Reports, 2014. 2(5): p. 592-605; Rimmbach, C., J. J. Jung, and R. David, Generation of Murine Cardiac Pacemaker Cell Aggregates Based on ES-Cell-Programming in Combination with Myh6-Promoter-Selection. J Vis Exp, 2015 (96); DE 10 2013 114 671 A1; WO 2015/091157 A1). Through detailed analysis by means of confocal microscopy, FACS, individual-cell patch clamp, funny channel density measurement and Ca²⁺ imaging, it was possible to show that iSABs consist of more than 80% terminally differentiated sinus node cells. The analysis of the remaining cells revealed that said cells are still incompletely differentiated sinus node cells. With the aid of an ex vivo model of ventricular slice cultures, it was possible to show the pacemaker function of the iSABs: they were capable of integrating into the slice and of inducing robust synchronous contractions thereof iSABs thus represent the first example of high-purity in vitro generated, functional sinus node tissue. Transferability from the murine system to the human system is possible, since the underlying processes are highly conserved.

The method according to the invention makes it possible to investigate conduction ex vivo or in vitro on a model system. Here, it is possible, if desired, to work with specific test subjects, since it is, for example, possible to use working-myocardium cells from a test subject. Conduction can be measured by means of ECG, for example using a so-called mini-ECG, or by means of microelectrode array (MEA). “Conduction” is to be understood here to mean the passing-on of electrical impulses from the pacemaker, in this case the iSABs, especially the sinus node cells of the iSABs, to the working-myocardium cells. The iSABs are, as described above, produced according to Jung, J. J., et al., Programming and isolation of highly pure physiologically and pharmacologically functional sinus-nodal bodies from pluripotent stem cells. Stem Cell Reports, 2014. 2(5): p. 592-605; Rimmbach, C., J. J. Jung, and R. David, Generation of Murine Cardiac Pacemaker Cell Aggregates Based on ES-Cell-Programming in Combination with Myh6-Promoter-Selection. J Vis Exp, 2015(96); DE 10 2013 114 671 A1 or WO 2015/091157 A1.

Preferably, the method according to the invention further comprises a comparison of the cardiac conduction measured in (iii) with a reference value, the comparison preferably comprising
(iv) administering a reference substance;
(v) measuring the cardiac conduction in the presence of the reference substance according to (iv);
(vi) comparing the values measured according to (iii) and the values measured according to (v).

For the comparison according to (vi), a change in the cardiac conduction of (v) in comparison with the value measured according to (iii) can be considered significant when the value of the cardiac conduction of (v) diverges by at least 5% from the value measured according to (iii). Preferably, a deviation by at least 10%, further preferably by at least 20%, is rated as significant. Preferably, the method comprises (i), (ii), (iii), (iv), (v) and (vi). In a preferred embodiment, the method consists of (i), (ii), (iii), (iv), (v) and (vi).

In principle, there are no restrictions with respect to the nature of the reference substance. Preferably, the reference substance is selected from the group of heart rate-lowering substances or from the group of heart rate-raising substances, preferably from the group consisting of isoprenaline, ZD-7288, zatebradine and ivabradine.

In a preferred embodiment, the method further comprises
(vii) washing out the reference substance according to (iv), (v);
(viii) measuring the cardiac conduction;
(ix) optionally comparing the values measured according to (viii) and the values measured according to (iii) or according to (v).

For the comparison according to (ix), a change in the cardiac conduction of (viii) in comparison with the value measured according to (iii) or according to (v) can be considered significant when the value of the cardiac conduction of (viii) diverges by at least 5% from the value measured according to (iii) or according to (v). Preferably, a deviation by at least 10%, further preferably by at least 20%, is rated as significant. Preferably, the method comprises (i), (ii), (iii), (iv), (v), (vi), (vii) and (viii), further preferably (i), (ii), (iii), (iv), (v), (vi), (vii), (viii) and (ix). In a preferred embodiment, the method consists of (i), (ii), (iii), (iv), (v), (vi), (vii) and (viii), further preferably of (i), (ii), (iii), (iv), (v), (vi), (vii), (viii) and (ix).

The working-myocardium cardiomyocytes according to (ii) preferably comprise atrial cells or ventricular cells or atrial and ventricular cells, further preferably (ii.1) atrial and (ii.2) ventricular cells, preference being given to the induced sinoatrial bodies according to (i), the atrial cells according to (ii.1) and the ventricular cells according to (ii.2) being arranged in a spatially separated manner, but in conductive communication with one another. Preferably, the spatial sequence is first (i), then (ii.1) and then (ii.2), i.e., the induced sinoatrial bodies (iSABs) comprising cardiac pacemaker cells according to (i) are arranged in a spatially separated manner, but in conductive communication with the atrial cells according to (ii.1), which in turn are arranged in a spatially separated manner, but in conductive communication with the ventricular cells according to (ii.2). “Arranged in conductive communication with one another” likewise means here that the passing-on of electrical impulses from the induced sinoatrial bodies according to (i) to the atrial cells according to (ii.1) and from said cells to the ventricular cells according to (ii.2) is possible despite spatial separation, there preferably being a spatially limited, direct contact between at least parts of the iSABs according to (i) and at least parts of the atrial cells according to (ii.1) and also between at least parts of the atrial cells according to (ii.1) and at least parts of the ventricular cells according to (ii.2), via which contact it is further preferably possible for connexin connections to form. “Spatially limited, direct contact” means that the spatially separated regions in which the iSABs according to (i) and the working-myocardium cardiomyocytes according to (ii) are situated have a common open contact area or a contact region via which the iSABS and the atrial cells, or the atrial cells and the ventricular cells, can come into contact.

In a preferred embodiment, the method is carried out in vitro.

The induced sinoatrial bodies (iSABs) comprising pacemaker cells are generated from multipotent or pluripotent stem cells, preferably from pluripotent stem cells. In a preferred embodiment, the induced sinoatrial bodies (iSABs) comprising pacemaker cells are generated from nonhuman embryonic stem cells or nonhuman induced pluripotent stem cells or human induced pluripotent stem cells or parthenogenetic stem cells or spermatogonial stem cells, preferably from nonhuman embryonic stem cells or nonhuman induced pluripotent stem cells or human induced pluripotent stem cells. In principle, the generation of the iSABs is not restricted to any particular method; for example, it can be achieved as described in DE 10 2013 114 671 A1 or WO 2015/091157 A1, i.e., iSABs can, for example, be reprogrammed from pluripotent stem cells into aggregates of cardiomyocytes with the aid of the combination of overexpression of TBX3 and an Myh6 promoter-based antibiotic selection.

### Determination of the Effect of Substances on Cardiac Conduction

The invention further relates to a method for determining the effect of substances on cardiac conduction, especially on the cardiac conduction of a test subject, comprising:
(i) providing induced sinoatrial bodies (iSABs) comprising cardiac pacemaker cells;
(ii) providing working-myocardium cardiomyocytes, especially working-myocardium cardiomyocytes of the test subject;
(iii) optionally measuring the cardiac conduction;
(iv) administering a substance to be investigated;
(v) measuring the cardiac conduction in the presence of the substance to be investigated according to (iv);
(vi) optionally comparing the cardiac conduction measured according to (iii) and the cardiac conduction measured according to (v);
the induced sinoatrial bodies according to (i) and the working-myocardium cardiomyocytes according to (ii) being arranged in a spatially separated manner, but in conductive communication with one another.

Preferably, the method for determining the effect of substances on cardiac conduction comprises (i), (ii), (iii), (iv) and (v), further preferably (i), (ii), (iii), (iv), (v) and (vi). In a preferred embodiment, the method for determining the effect of substances on cardiac conduction consists of (i), (ii), (iv) and (v), further preferably of (i), (ii), (iii), (iv) and (v), further preferably of (i), (ii), (iii), (iv), (v) and (vi).

“Arranged in conductive communication with one another” means that the passing-on of electrical impulses, i.e., the forwarding of the stimulus, for example from the induced sinoatrial bodies according to (i) to the working-myocardium cardiomyocytes according to (ii), is possible despite spatial separation, there preferably being a spatially limited, direct contact between at least parts of the iSABs according to (i) and at least parts of the working-myocardium cardiomyocytes according to (ii), via which contact it is further preferably possible for connexin connections to form between the iSABs according to (i) and the working-myocardium cardiomyocytes according to (ii). “Spatially limited, direct contact” means that the spatially separated regions in which the iSABs according to (i) and the working-myocardium cardiomyocytes according to (ii) are situated have a common open contact area or a contact region via which the iSABS and the working-myocardium cardiomyocytes can come into contact.

For the comparison according to (vi), a change in the cardiac conduction of (v), for example a change in the ECG-measured frequency of (v), in comparison with the value measured according to (iii) can be considered significant when the value of the cardiac conduction of (v) diverges by at least 5% from the value measured according to Preferably, a deviation by at least 10%, further preferably by at least 20%, is rated as significant.

In a preferred embodiment, the method further comprises
(vii) washing out the substance to be investigated according to (iv), (v);
(viii) measuring the cardiac conduction;
(ix) optionally comparing the values measured according to (viii) and the values measured according to (v) or according to (iii) or comparing the values measured according to (viii) and the values measured according to (v) and according to (iii).

For the comparison according to (ix), a change in the cardiac conduction of (viii) in comparison with the value measured according to (iii) and/or according to (v) can be considered significant when the value of the cardiac conduction of (viii) diverges by at least 5% from the value measured according to (iii) and/or according to (v). Preferably, a deviation by at least 10%, further preferably by at least 20%, is rated as significant. Preferably, the method for determining the effect of substances on cardiac conduction comprises (i), (ii), (iii), (iv), (v), (vii) and (viii), further preferably (i), (ii), (iii), (iv), (v), (vi), (vii) and (viii), further preferably (i), (ii), (iii), (iv), (v), (vi), (vii), (viii) and (ix). In a preferred embodiment, the method for determining the effect of substances on cardiac conduction consists of (i), (ii), (iii), (iv), (v), (vii) and (viii), further preferably of (i), (ii), (iii), (iv), (v), (vi), (vii) and (viii), further preferably of (i), (ii), (iii), (iv), (v), (vi), (vii), (viii) and (ix).

Preferably, the working-myocardium cardiomyocytes according to (ii) comprised atrial cells or ventricular cells or atrial and ventricular cells, preferably (ii.1) atrial and (ii.2) ventricular cells, preference being given to the induced sinoatrial bodies according to (i), the atrial cells according to (ii.1) and the ventricular cells according to (ii.2) being arranged in a spatially separated manner, but in conductive communication with one another. Preferably, the spatial sequence is first (i), then (ii.1) and then (ii.2), i.e., the induced sinoatrial bodies (iSABs) comprising cardiac pacemaker cells according to (i) are arranged in a spatially separated manner, but in conductive communication with the atrial cells according to (ii.1), which in turn are arranged in a spatially separated manner, but in conductive communication with the ventricular cells according to (ii.2). “Arranged in conductive communication with one another” likewise means here that the passing-on of electrical impulses from the induced sinoatrial bodies according to (i) to the atrial cells according to (ii.1) and from said cells to the ventricular cells according to (ii.2) is possible despite spatial separation, there preferably being a spatially limited, direct contact between at least parts of the iSABs according to (i) and at least parts of the atrial cells according to (ii.1) and also between at least parts of the atrial cells according to (ii.1) and at least parts of the ventricular cells according to (ii.2), via which contact it is further preferably possible for connexin connections to form. “Spatially limited, direct contact” likewise means here that the spatially separated regions in which the iSABs according to (i) and atrial cells according to (ii.1) are situated have a common open contact area or a contact region via which the iSABs and the atrial cells can come into contact and that the regions in which the atrial cells according to (ii.1) and the ventricular cells according to (ii.2) are situated have a common open contact area or a contact region via which the atrial cells and the ventricular cells can come into contact.

Preferably, the method is carried out in vitro.

The induced sinoatrial bodies (iSABs) comprising pacemaker cells are generated from multipotent or pluripotent stem cells, preferably from pluripotent stem cells. In a preferred embodiment, the induced sinoatrial bodies (iSABs) comprising pacemaker cells are generated from nonhuman embryonic stem cells or nonhuman induced pluripotent stem cells or human induced pluripotent stem cells or parthenogenetic stem cells or spermatogonial stem cells, preferably from nonhuman embryonic stem cells or nonhuman induced pluripotent stem cells or human induced pluripotent stem cells. In principle, the generation of the iSABs is not restricted to any particular method; for example, it can be achieved as described in DE 10 2013 114 671 A1 or WO 2015/091157 A1, i.e., iSABs can, for example, be reprogrammed from pluripotent stem cells into aggregates of cardiomyocytes with the aid of the combination of overexpression of TBX3 and an Myh6 promoter-based antibiotic selection.

Identification of Substances which have an Effect on Cardiac Conduction

The invention further relates to a method for identifying substances which have an effect on cardiac conduction, especially on the cardiac conduction of a test subject, comprising:
(i) providing induced sinoatrial bodies (iSABs) comprising pacemaker cells;
(ii) providing working-myocardium cardiomyocytes, especially working-myocardium cardiomyocytes of the test subject;
(iii) optionally measuring the cardiac conduction;
(iv) administering a substance to be investigated;
(v) measuring the cardiac conduction;
(vi) optionally comparing the cardiac conduction measured according to (iii) and the cardiac conduction measured according to (v); and optionally determining whether the substance to be investigated has an effect on cardiac conduction on the basis of the comparison according to (vi);
the induced sinoatrial bodies according to (i) and the working-myocardium cardiomyocytes according to (ii) being arranged in a spatially separated manner, but in conductive communication with one another.

Preferably, the method for identifying substances which have an effect on cardiac conduction comprises (i), (ii), (iii), (iv) and (v), further preferably (i), (ii), (iii), (iv), (v) and (vi). In a preferred embodiment, the method for identifying substances which have an effect on cardiac conduction consists of (i), (ii), (iv) and (v), further preferably of (i), (ii), (iii), (iv) and (v), further preferably of (i), (ii), (iii), (iv), (v) and (vi).

“Arranged in conductive communication with one another” means that the passing-on of electrical impulses, i.e., the forwarding of the stimulus, for example from the induced sinoatrial bodies according to (i) to the working-myocardium cardiomyocytes according to (ii), is possible despite spatial separation, there preferably being a spatially limited, direct contact between at least parts of the iSABs according to (i) and at least parts of the working-myocardium cardiomyocytes according to (ii), via which contact it is further preferably possible for connexin connections to form between the iSABs according to (i) and the working-myocardium cardiomyocytes according to (ii). “Spatially limited, direct contact” means that the spatially separated regions in which the iSABs according to (i) and the working-myocardium cardiomyocytes according to (ii) are situated have a common open contact area or a contact region via which the iSABS and the working-myocardium cardiomyocytes can come into contact.

For the comparison according to (vi), a change in the cardiac conduction of (v) in comparison with the value measured according to (iii) can be considered significant when the value of the cardiac conduction of (viii) diverges by at least 5% from the value measured according to (iii). Preferably, a deviation by at least 10%, further preferably by at least 20%, is rated as significant.

In a preferred embodiment, the method further comprises
(vii) washing out the substance to be investigated according to (iv)
(vii) measuring the cardiac conduction;
(viii) optionally comparing the values measured according to (vii) and the values measured according to (iii) or according to (v); or comparing the values measured according to (vii) and the values measured according to (iii) and the values measured according to (v);
(ix) determining whether the substance to be investigated has an effect on cardiac conduction on the basis of the comparison according to (viii).

For the comparison according to (ix), a change in the cardiac conduction of (viii) in comparison with the value measured according to (iii) and/or according to (v) can be considered significant when the value of the cardiac conduction of (viii) diverges by at least 5% from the value measured according to (iii) and/or according to (v). Preferably, a deviation by at least 10%, further preferably by at least 20%, is rated as significant. Preferably, the method for identifying substances which have an effect on cardiac conduction comprises (i), (ii), (iii), (iv), (v), (vii) and (ix), further preferably (i), (ii), (iii), (iv), (v), (vi), (vii) and (ix), further preferably (i), (ii), (iii), (iv), (v), (vi), (vii), (viii) and (ix). In a preferred embodiment, the method for identifying substances which have an effect on cardiac conduction consists of (i), (ii), (iii), (iv), (v), (vii) and (ix), further preferably of (i), (ii), (iii), (iv), (v), (vi), (vii) and (ix), further preferably of (i), (ii), (iii), (iv), (v), (vi), (vii), (viii) and (ix).

Preferably, the working-myocardium cardiomyocytes according to (ii) comprise atrial cells or ventricular cells or atrial and ventricular cells, preferably (ii.1) atrial and (ii.2) ventricular cells, preference being given to the induced sinoatrial bodies according to (i), the atrial cells according to (ii.1) and the ventricular cells according to (ii.2) being arranged in a spatially separated manner, but in conductive communication with one another. Preferably, the spatial sequence is first (i), then (ii.1) and then (ii.2), i.e., the induced sinoatrial bodies (iSABs) comprising cardiac pacemaker cells according to (i) are arranged in a spatially separated manner, but in conductive communication with the atrial cells according to (ii.1), which in turn are arranged in a spatially separated manner, but in conductive communication with the ventricular cells according to (ii.2). “Arranged in conductive communication with one another” likewise means here that the passing-on of electrical impulses from the induced sinoatrial bodies according to (i) to the atrial cells according to (ii.1) and from said cells to the ventricular cells according to (ii.2) is possible despite spatial separation, there preferably being a spatially limited, direct contact between at least parts of the iSABs according to (i) and at least parts of the atrial cells according to (ii.1) and also between the atrial cells according to (ii.1) and the ventricular cells according to (ii.2), via which contact it is further preferably possible for connexin connections to form. “Spatially limited, direct contact” likewise means here that the spatially separated regions in which the iSABs according to (i) and atrial cells according to (ii.1) are situated have a common open contact area or a contact region via which the iSABs and the atrial cells can come into contact and that the regions in which the atrial cells according to (ii.1) and the ventricular cells according to (ii.2) are situated have a common open contact area or a contact region via which the atrial cells and the ventricular cells can come into contact.

Preferably, the method is carried out in vitro.

The induced sinoatrial bodies (iSABs) comprising pacemaker cells are generated from multipotent or pluripotent stem cells, preferably from pluripotent stem cells. In a preferred embodiment, the induced sinoatrial bodies (iSABs) comprising pacemaker cells are generated from nonhuman embryonic stem cells or nonhuman induced pluripotent stem cells or human induced pluripotent stem cells or parthenogenetic stem cells or spermatogonial stem cells, preferably from nonhuman embryonic stem cells or nonhuman induced pluripotent stem cells or human induced pluripotent stem cells. In principle, the generation of the iSABs is not restricted to any particular method; for example, it can be achieved as described in DE 10 2013 114 671 A1 or WO 2015/091157 A1, i.e., iSABs can, for example, be reprogrammed from pluripotent stem cells into aggregates of cardiomyocytes with the aid of the combination of overexpression of TBX3 and an Myh6 promoter-based antibiotic selection.

### Device for Determining a Cardiac Conduction

The invention further relates to a device for determining a cardiac conduction, especially a cardiac conduction of a test subject, comprising:
(a) a first region 1 suitable for accommodating induced sinoatrial bodies (iSABs) comprising pacemaker cells;
(b) a second region 2 suitable for accommodating working-myocardium cardiomyocytes, especially working-myocardium cardiomyocytes of the test subject;
(c) a third region 3 which is suitable for allowing a conduction from the first region 1 into the second region 2.

“Suitable for allowing a conduction from the first region 1 into the second region 2” means that the first region and the second region are in conductive communication, i.e., can communicate in a conductive manner, via the third region. “In conductive communication” means that the passing-on of electrical impulses, i.e., the forwarding of the stimulus, for example from the first region into the second region via the third region, is possible despite spatial separation, the third region preferably being a spatially limited, direct contact area or contact region between first and second region. In the case of the presence of iSABs and working-myocardium cardiomyocytes, this means that at least parts of the iSABs and at least parts of the working-myocardium cardiomyocytes can come into contact via the third region, further preferably via the formation of connexin connections between the spatially separated iSABs and the working-myocardium cardiomyocytes. The third region can be a contact area, i.e., first and second region have a common open contact area. Alternatively, the third region can comprise a spatial extent along the axis of first and second region.

In principle, the device is not subject to any restrictions with respect to its structure. By way of example, one possible structure are two recesses in a plate, the first recess and the second recess being respectively the first region 1 according to (a) and the second region 2 according to (b), which are connected via a third recess (3rd region 3 according to (c)), the depth of which is the same as or less than the minimum depth of the first or second recess. A corresponding structure is shown schematically in FIG. 1A. It would be possible to establish conductive communication by contact of iSABs from the first region 1 with working-myocardium cardiomyocytes from the second region 2 in the third region 3t. Here, the third region is a direct contact area. Alternatively, it would be possible for the third region to comprise a spatial extent along the axis of first and second region, the depth of the third region or of the corresponding recess likewise being the same as or less than the minimum depth of the first or second recess. Here, the conductive communication is achieved by an extension of iSABs from the first region into the third region and by an extension of working-myocardium cardiomyocytes from the second region into the third region, this making it possible in the third region to establish contact of iSABs from the first region with working-myocardium cardiomyocytes from the second region. A corresponding structure is shown schematically in FIG. 1B.

In one embodiment, the device for determining a cardiac conduction, especially a cardiac conduction of a test subject, comprises:
(a) a first region 1 containing induced sinoatrial bodies (iSABs) comprising pacemaker cells;
(b) a second region 2 suitable for accommodating working-myocardium cardiomyocytes, especially working-myocardium cardiomyocytes of the test subject;
(c) a third region 3 which is suitable for allowing a conduction from the first region 1 into the second region 2.

“Suitable for allowing a conduction from the first region 1 into the second region 2” also means here that the first region and the second region are in conductive communication, i.e., can communicate in a conductive manner, via the third region. “In conductive communication” means that the passing-on of electrical impulses, i.e., the forwarding of the stimulus, for example from the first region into the second region via the third region, is possible despite spatial separation. With respect to the structure of the device, what has been mentioned above applies.

In a preferred embodiment, the second region 2 of the device according to (b) comprises:
(b.1) a region 2.1 which is suitable for accommodating atrial cells;
(b.2) a region 2.2 which is suitable for accommodating ventricular cells;
the third region 3 according to (c) comprising:
(c.1) a region 3.1 which is suitable for allowing a conduction from the first region 1 according to (a) into the region 2.1 according to (b.1);
(c.2) a region 3.2 which is suitable for allowing a conduction from the region 2.1 according to (b.1) into the region 2.2 according to (b.2).

“Suitable for allowing a conduction from the first region 1 into the region 2.1 according to (b.1) and a conduction from region 2.1 according to (b.1) into the region 2.2 according to (b.2)” also means here that the first region and the region 2.1 are in conductive communication, i.e., can communicate in a conductive manner, via the region 3.1 and that the region 2.1 and the region 2.2 are in conductive communication, i.e., can communicate in a conductive manner, via the region 3.2. “Suitable for allowing a conduction from the first region into the second region” means that the first region and the second region are in conductive communication, i.e., can communicate in a conductive manner, via the third region. “In conductive communication” means that the passing-on of electrical impulses, i.e., the forwarding of the stimulus, from the first region 1 into the region 2.1 via the region 3.1 and, further, from the region 2.1 into the region 2.2 via the region 3.2 is possible despite spatial separation. With respect to the structure of the device and the regions 1, 2.1, 2.2, 3.1 and 3.2, what has been mentioned at the start in relation to the structure of the device likewise applies. A corresponding structure is shown schematically in FIGS. 2A and 2B.

In principle, there is no restriction with respect to the material of which the device consists. Preferably, the device consists of a material selected from the group consisting of glass, ceramic, plastic and a mixture of two or more of these materials, the plastic preferably being selected from the group consisting of polystyrene, polyethylene, polypropylene, polyethylene terephthalate and mixtures of two or more of these substances, preferably polystyrene.

The induced sinoatrial bodies (iSABs) comprising pacemaker cells are generated from multipotent or pluripotent stem cells, preferably from pluripotent stem cells. Preferably, the induced sinoatrial bodies (iSABs) comprising pacemaker cells are generated from nonhuman embryonic stem cells or nonhuman induced pluripotent stem cells or human induced pluripotent stem cells or parthenogenetic stem cells or spermatogonial stem cells, preferably from nonhuman embryonic stem cells or nonhuman induced pluripotent stem cells or human induced pluripotent stem cells.

### Kit for Determining a Cardiac Conduction

The invention further relates to a kit for determining a cardiac conduction, especially a cardiac conduction of a test subject, comprising

(A) a device comprising:
(a) a first region 1 suitable for accommodating induced sinoatrial bodies (iSABs);
(b) a second region 2 suitable for accommodating working-myocardium cardiomyocytes, especially working-myocardium cardiomyocytes of the test subject;
(c) a third region 3 which is suitable for allowing a conduction from the first region 1 into the second region 2;

(B) induced sinoatrial bodies (iSABs) comprising pacemaker cells.

“Suitable for allowing a conduction from the first region 1 into the second region 2” also means here that the first region and the second region are in conductive communication, i.e., can communicate in a conductive manner, via the third region. “In conductive communication” means that the passing-on of electrical impulses, i.e., the forwarding of the stimulus, for example from the first region into the second region via the third region, is possible despite spatial separation.

In principle, the device is not subject to any restrictions with respect to its structure. By way of example, a possible structure are two recesses in a plate, the first recess and the second recess being respectively the first region 1 according to (a) and the second region 2 according to (b), which are connected via a third recess (3rd region 3 according to (c)), the depth of which is the same as or less than the minimum depth of the first or second recess. A corresponding structure is shown schematically in FIG. 1A. It would be possible to establish conductive communication by contact of iSABs from the first region 1 with working-myocardium cardiomyocytes from the second region 2 in the third region 3. Here, the third region is a direct contact area. Alternatively, it would be possible for the third region to comprise a spatial extent along the axis of first and second region, the depth of the third region or of the corresponding recess likewise being the same as or less than the minimum depth of the first or second recess. Here, the conductive communication is achieved by an extension of iSABs from the first region into the third region and by an extension of working-myocardium cardiomyocytes from the second region into the third region, this making it possible in the third region to establish contact of iSABs from the first region with working-myocardium cardiomyocytes from the second region. A corresponding structure is shown schematically in FIG. 1B.

In a preferred embodiment of the kit, the second region 2 according to (b) comprises:

(b.1) a region 2.1 which is suitable for accommodating atrial cells;

(b.2) a region 2.2 which is suitable for accommodating ventricular cells;

the third region (c) comprising:

(c.1) a region 3.1 which is suitable for allowing a conduction from the first region 1 according to (a) into the region 2.1 according to (b.1);

(c.2) a region 3.2 which is suitable for allowing a conduction from the region 2.1 according to (b.1) into the region 2.2 according to (b.2).

“Suitable for allowing a conduction from the first region 1 into the region 2.1 according to (b.1) and a conduction from region 2.1 according to (b.1) into the region 2.2 according to (b.2)” also means here that the first region and the region 2.1 are in conductive communication, i.e., can communicate in a conductive manner, via the region 3.1 and that the region 2.1 and the region 2.2 are in conductive communication, i.e., can communicate in a conductive manner, via the region 3.2. “In conductive communication” means that the passing-on of electrical impulses, i.e., the forwarding of the stimulus, from the region first region 1 into the region 2.1 via the region 3.1 and, further, from the region 2.1 into the region 2.2 via the region 3.2 is possible despite spatial separation. With respect to the structure of the device and the regions 1, 2.1, 2.2, 3.1 and 3.2, what has been mentioned at the start in relation to the structure of the device likewise applies. The corresponding structure is shown schematically in FIGS. 2A and 2B.

In principle, there is no restriction with respect to the material of which the device of the kit consists. Preferably, the device consists of a material selected from the group consisting of glass, ceramic, plastic and a mixture of two or more of these materials, the plastic preferably being selected from the group consisting of polystyrene, polyethylene, polypropylene, polyethylene terephthalate and mixtures of two or more of these substances, preferably polystyrene.

The induced sinoatrial bodies (iSABs) comprising pacemaker cells are generated from multipotent or pluripotent stem cells, preferably from pluripotent stem cells. Preferably, the induced sinoatrial bodies (iSABs) comprising pacemaker cells are generated from nonhuman embryonic stem cells or nonhuman induced pluripotent stem cells or human induced pluripotent stem cells or parthenogenetic stem cells or spermatogonial stem cells, preferably from nonhuman embryonic stem cells or nonhuman induced pluripotent stem cells or human induced pluripotent stem cells.

### Use

In principle, there are no restrictions as to what the device and the kit are used for. Preferably, the device or the kit is used for the in vitro evaluation of drugs.

In a preferred embodiment, the device or the kit is used for identifying substances which have an effect on cardiac conduction.

In a further preferred embodiment, the device or the kit is for the diagnosis or prediagnosis, especially for the in vitro diagnosis or in vitro prediagnosis, of a disturbed cardiac conduction in a test subject or for the determination of the risk of a test subject developing a disturbed cardiac conduction.

The present invention is illustrated in more detail by the following embodiments and combinations of embodiments that arise from the corresponding dependency references and other references. It should be noted here that, in all cases in which a range of embodiments is mentioned, for example in the context of an expression such as “method according to any of embodiments 1 to 4”, each embodiment in this range is to be considered explicitly disclosed to a person skilled in the art, i.e., said expression is to be understood by a person skilled in the art as synonymous with “method according to any of (each of) embodiments 1, 2, 3 and 4”.

### Embodiments

1. A method for determining cardiac conduction, especially the cardiac conduction of a test subject, comprising:
   (i) providing induced sinoatrial bodies (iSABs) comprising cardiac pacemaker cells;
   (ii) providing working-myocardium cardiomyocytes, especially working-myocardium cardiomyocytes of the test subject;
   (iii) measuring the cardiac conduction;
   the induced sinoatrial bodies according to (i) and the working-myocardium cardiomyocytes according to (ii) being arranged in a spatially separated manner, but in conductive communication with one another.
2. The method according to embodiment 1, comprising
   comparison of the cardiac conduction measured in (iii) with a reference value, the comparison preferably comprising
   (iv) administering a reference substance;
   (v) measuring the cardiac conduction in the presence of the reference substance according to (iv);
   (vi) comparing the values measured according to (iii) and the values measured according to (v).
3. The method according to embodiment 2, wherein the reference substance is selected from the group of heart rate-lowering substances or from the group of heart rate-raising substances, preferably from the group consisting of isoprenaline, ZD-7288 zatebradine and ivabradine.
4. The method according to any of embodiments 1 to 3, comprising
   (vii) washing out the reference substance according to (iv), (v);
   (viii) measuring the cardiac conduction;
   (ix) optionally comparing the values measured according to (viii) and the values measured according to (iii) or according to (v).
5. The method according to any of embodiments 1 to 4, wherein the working-myocardium cardiomyocytes according to (ii) comprise atrial cells or ventricular cells or atrial and ventricular cells, preferably (ii.1) atrial and (ii.2) ventricular cells, preference being given to the induced sinoatrial bodies according to (i), the atrial cells according to (ii.1) and the ventricular cells according to (ii.2) being arranged in a spatially separated manner, but in conductive communication with one another.
6. The method according to any of embodiments 1 to 5, which is carried out in vitro.
7. The method according to any of embodiments 1 to 6, wherein the induced sinoatrial bodies (iSABs) comprising pacemaker cells are generated from multipotent or pluripotent stem cells, preferably from pluripotent stem cells.
8. The method according to any of embodiments 1 to 7, wherein the induced sinoatrial bodies (iSABs) comprising pacemaker cells are generated from nonhuman embryonic stem cells or nonhuman induced pluripotent stem cells or human induced pluripotent stem cells or parthenogenetic stem cells or spermatogonial stem cells, preferably from nonhuman embryonic stem cells or nonhuman induced pluripotent stem cells or human induced pluripotent stem cells.
9. A method for determining the effect of substances on cardiac conduction, especially on the cardiac conduction of a test subject, comprising:
   (i) providing induced sinoatrial bodies (iSABs) comprising cardiac pacemaker cells;
   (ii) providing working-myocardium cardiomyocytes, especially working-myocardium cardiomyocytes of the test subject;
   (iii) optionally measuring the cardiac conduction;
   (iv) administering a substance to be investigated;
   (v) measuring the cardiac conduction in the presence of the substance to be investigated according to (iv);
   (vi) optionally comparing the cardiac conduction measured according to (iii) and the cardiac conduction measured according to (v);
   the induced sinoatrial bodies according to (i) and the working-myocardium cardiomyocytes according to (ii) being arranged in a spatially separated manner, but in conductive communication with one another.
10. The method according to embodiment 9, comprising
   (vii) washing out the substance to be investigated according to (iv), (v);
   (viii) measuring the cardiac conduction;
   (ix) optionally comparing the values measured according to (viii) and the values measured according to (iii) or according to (v); or comparing the values measured according to (viii) and the values measured according to (iii) and according to (v).
11. The method according to embodiment 9 to 10, wherein the working-myocardium cardiomyocytes according to (ii) comprise atrial cells or ventricular cells or atrial and ventricular cells, preferably (ii.1) atrial and (ii.2) ventricular cells, preference being given to the induced sinoatrial bodies according to (i), the atrial cells according to (ii.1) and the ventricular cells according to (ii.2) being arranged in a spatially separated manner, but in conductive communication with one another.
12. The method according to any of embodiments 9 to 11, which is carried out in vitro.
13. The method according to any of embodiments 9 to 12, wherein the induced sinoatrial bodies (iSABs) comprising pacemaker cells are generated from multipotent or pluripotent stem cells, preferably from pluripotent stem cells.
14. The method according to any of embodiments 9 to 13, wherein the induced sinoatrial bodies (iSABs) comprising pacemaker cells are generated from nonhuman embryonic stem cells or nonhuman induced pluripotent stem cells or human induced pluripotent stem cells or parthenogenetic stem cells or spermatogonial stem cells, preferably from nonhuman embryonic stem cells or nonhuman induced pluripotent stem cells or human induced pluripotent stem cells.
15. A method for identifying substances which have an effect on cardiac conduction, especially on the cardiac conduction of a test subject, comprising:
   (i) providing induced sinoatrial bodies (iSABs) comprising pacemaker cells;
   (ii) providing working-myocardium cardiomyocytes, especially working-myocardium cardiomyocytes of the test subject;
   (iii) optionally measuring the cardiac conduction;
   (iv) administering a substance to be investigated;
   (v) measuring the cardiac conduction;
   (vi) optionally comparing the cardiac conduction measured according to (iii) and the cardiac conduction measured according to (v); and optionally determining whether the substance to be investigated has an effect on cardiac conduction on the basis of the comparison according to (vi);
   the induced sinoatrial bodies according to (i) and the working-myocardium cardiomyocytes according to (ii) being arranged in a spatially separated manner, but in conductive communication with one another.
16. The method according to embodiment 15, comprising
   (vii) washing out the substance to be investigated according to (iv)
   (vii) measuring the cardiac conduction;
   (viii) optionally comparing the values measured according to (vii) and the values measured according to (iii) or according to (v); or comparing the values measured according to (vii) and the values measured according to (iii) and the values measured according to (v);
   (ix) optionally determining whether the substance to be investigated has an effect on cardiac conduction on the basis of the comparison according to (viii).
17. The method according to embodiment 16, wherein the working-myocardium cardiomyocytes according to (ii) comprise atrial cells or ventricular cells or atrial and ventricular cells, preferably (ii.1) atrial and (ii.2) ventricular cells, preference being given to the induced sinoatrial bodies according to (i), the atrial cells according to (ii.1) and the ventricular cells according to (ii.2) being arranged in a spatially separated manner, but in conductive communication with one another.
18. The method according to embodiment 16 or 17, which is carried out in vitro,
19. The method according to any of embodiments 16 to 18, wherein the induced sinoatrial bodies (iSABs) comprising pacemaker cells are generated from multipotent or pluripotent stem cells, preferably from pluripotent stem cells.
20. The method according to any of embodiments 16 to 19, wherein the induced sinoatrial bodies (iSABs) comprising pacemaker cells are generated from nonhuman embryonic stem cells or nonhuman induced pluripotent stem cells or human induced pluripotent stem cells or parthenogenetic stem cells or spermatogonial stem cells, preferably from nonhuman embryonic stem cells or nonhuman induced pluripotent stem cells or human induced pluripotent stem cells.
21. A device for determining a cardiac conduction, especially a cardiac conduction of a test subject, comprising:
   (a) a first region 1 suitable for accommodating induced sinoatrial bodies (iSABs) comprising pacemaker cells;
   (b) a second region 2 suitable for accommodating working-myocardium cardiomyocytes, especially working-myocardium cardiomyocytes of the test subject;
   (c) a third region 3 which is suitable for allowing a conduction from the first region 1 into the second region 2.
22. The device for determining a cardiac conduction, especially a cardiac conduction of a test subject, according to embodiment 21, comprising:
   (a) a first region 1 containing induced sinoatrial bodies (iSABs) comprising pacemaker cells;
   (b) a second region 2 suitable for accommodating working-myocardium cardiomyocytes, especially working-myocardium cardiomyocytes of the test subject;
   (c) a third region 3 which is suitable for allowing a conduction from the first region 1 into the second region 2.
23. The device according to embodiment 21 or according to embodiment 22, wherein the second region 2 according to (b) comprises:
   (b.1) a region 2.1 which is suitable for accommodating atrial cells;
   (b.2) a region 2.2 which is suitable for accommodating ventricular cells;
   the third region 3 according to (c) comprising:
   (c.1) a region 3.1 which is suitable for allowing a conduction from the first region 1 according to (a) into the region 2.1 according to (b.1);
   (c.2) a region 3.2 which is suitable for allowing a conduction from the region 2.1 according to (b.1) into the region 2.2 according to (b.2).
24. The device according to any of embodiments 21 to 22, wherein the device consists of a material selected from the group consisting of glass, ceramic, plastic and a mixture of two or more of these materials, the plastic preferably being selected from the group consisting of polystyrene, polyethylene, polypropylene, polyethylene terephthalate and mixtures of two or more of these substances, preferably polystyrene.
25. The device according to any of embodiments 21 to 24, wherein the induced sinoatrial bodies (iSABs) comprising pacemaker cells are generated from multipotent or pluripotent stem cells, preferably from pluripotent stem cells.
26. The device according to any of embodiments 21 to 25, wherein the induced sinoatrial bodies (iSABs) comprising pacemaker cells are generated from nonhuman embryonic stem cells or nonhuman induced pluripotent stem cells or human induced pluripotent stem cells or parthenogenetic stem cells or spermatogonial stem cells, preferably from nonhuman embryonic stem cells or nonhuman induced pluripotent stem cells or human induced pluripotent stem cells.
27. A kit for determining a cardiac conduction, especially a cardiac conduction of a test subject, comprising
   (A) a device comprising:
      (a) a first region 1 suitable for accommodating induced sinoatrial bodies (iSABs);
      (b) a second region 2 suitable for accommodating working-myocardium cardiomyocytes, especially working-myocardium cardiomyocytes of the test subject;
      (c) a third region 3 which is in conductive communication with the first and the second region and is suitable for allowing a conduction from the first region 1 into the second region 2;
   (B) induced sinoatrial bodies (iSABs) comprising pacemaker cells.
28. The kit according to embodiment 27, wherein the second region 2 according to (b) comprises:
   (b.1) a region 2.1 which is suitable for accommodating atrial cells;
   (b.2) a region 2.2 which is suitable for accommodating ventricular cells;
   the third region 3 according to (c) comprising:
   (c.1) a region 3.1 which is suitable for allowing a conduction from the first region 1 according to (a) into the region 2.1 according to (b.1);
   (c.2) a region 3.2 which is suitable for allowing a conduction from the region 2.1 according to (b.1) into the region 2.2 according to (b.2).
29. The kit according to embodiment 27 or 28, wherein the device consists of a material selected from the group consisting of glass, ceramic, plastic and a mixture of two or more of these materials, the plastic preferably being selected from the group consisting of polystyrene, polyethylene, polypropylene, polyethylene terephthalate and mixtures of two or more of these substances, preferably polystyrene.
30. The kit according to any of embodiments 27 to 29, wherein the induced sinoatrial bodies (iSABs) comprising pacemaker cells are generated from multipotent or pluripotent stem cells, preferably from pluripotent stem cells.
31. The kit according to any of embodiments 27 to 30, wherein the induced sinoatrial bodies (iSABs) comprising pacemaker cells are generated from nonhuman embryonic stem cells or nonhuman induced pluripotent stem cells or human induced pluripotent stem cells or parthenogenetic stem cells or spermatogonial stem cells, preferably from nonhuman embryonic stem cells or nonhuman induced pluripotent stem cells or human induced pluripotent stem cells.
32. The use of a device according to any of embodiments 21 to 26 or of the kit according to any of embodiments 27 to 31 for the in vitro evaluation of drugs.
33. The use of a device according to any of embodiments 21 to 26 or of the kit according to any of embodiments 27 to 31 for the identification of substances which have an effect on cardiac conduction.
34. The use of a device according to any of embodiments 21 to 26 or of the kit according to any of embodiments 27 to 31 for the diagnosis or prediagnosis, especially for the in vitro diagnosis or in vitro prediagnosis, of a disturbed cardiac conduction in a test subject or for the determination of the risk of a test subject developing a disturbed cardiac conduction.

### BRIEF DESCRIPTION OF THE FIGURES

FIG. 1 shows schematically the structure of a device having a first region (1) suitable for accommodating induced sinoatrial bodies (iSABs); a second region (2) suitable for accommodating working-myocardium cardiomyocytes; and a third region (3) which is in conductive communication with the first and the second region and is suitable for allowing a conduction from the first region into the second region; in FIG. 1A, the third region (3) is a direct contact region between first region (1) and second region (2); in FIG. 1B, the third region (3) comprises a spatial extent along the axis of first and second region.

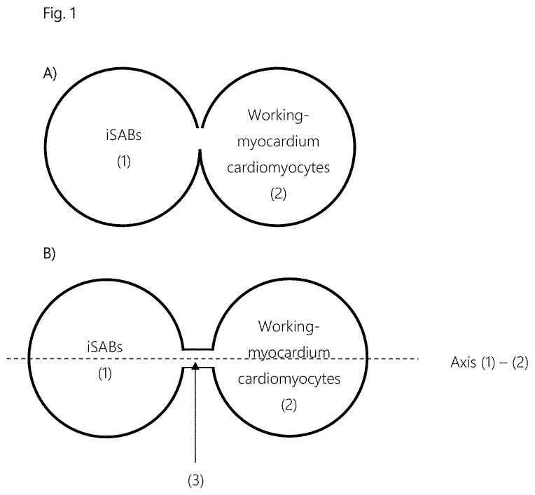

FIG. 2 shows schematically the structure of a device having a first region (1) suitable for accommodating induced sinoatrial bodies (iSABs); a second region (2) suitable for accommodating working-myocardium cardiomyocytes; and a third region (3), the region (2) comprising two regions 2.1 and 2.2, in which 2.1 is suitable for accommodating atrial cells and 2.2 is suitable for accommodating ventricular cells; the third region (3) comprises a region 3.1 and a region 3.2; in FIG. 2A, the third region (3.1) is a direct contact region between 1.1 and 2.1, and additionally 3.2 is a direct contact region between 2.1 and 2.2; in FIG. 2B, 3.1 and 3.2 comprise in both cases a spatial extent along the axis of regions (1), (2.1), (2.2).

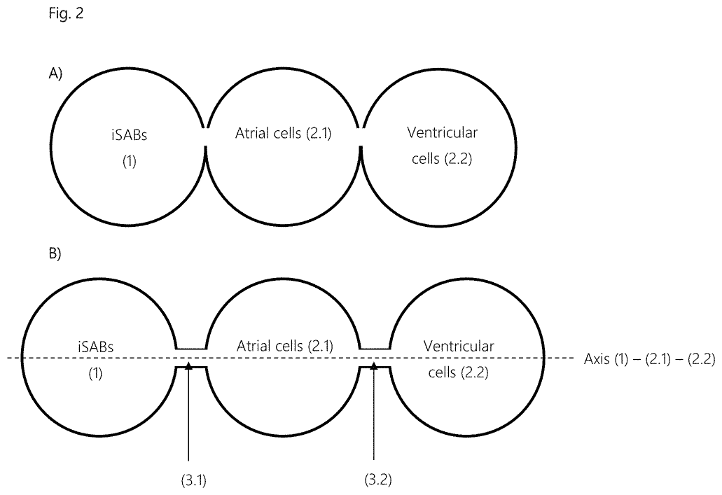

FIG. 3 shows a prototype of a synchronized conductoid composed of murine iSABs coupled to murine ventricular cardiomyocytes; FIG. 3B shows the iSABs and ventricular cardiomyocytes, the iSABs (dotted border) being capable of coupling to ventricular cardiomyocytes (hatched arrow) and of inducing rhythmically synchronized contractions thereof with ˜300 bpm; FIG. 3A shows the corresponding deflections.

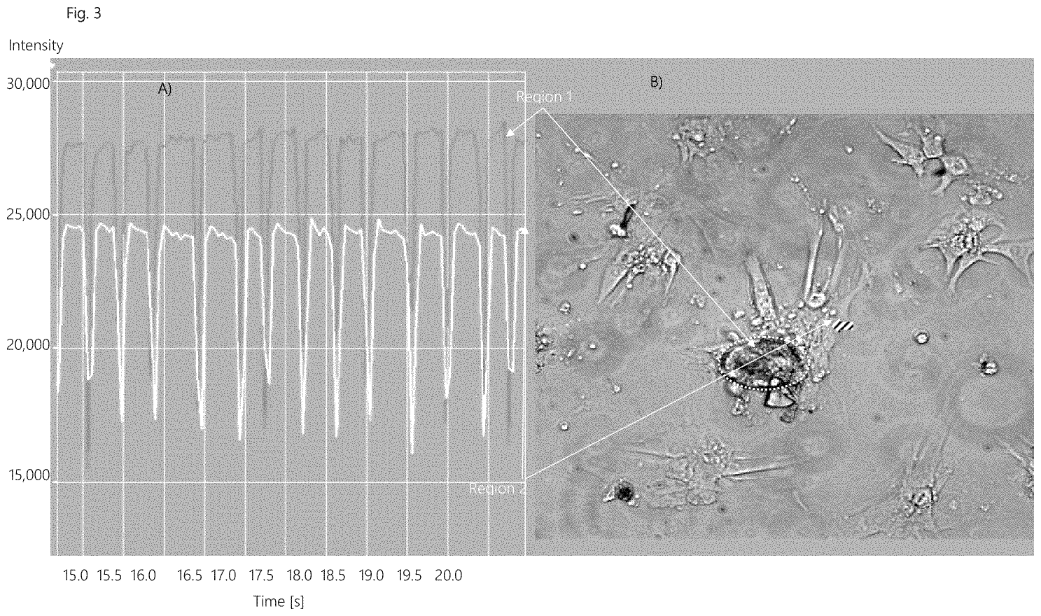

### CITED LITERATURE

Jung, J. J., et al., Programming and isolation of highly pure physiologically and pharmacologically functional sinus-nodal bodies from pluripotent stem cells. Stem Cell Reports, 2014. 2(5): p. 592-605
Rimmbach, C., J. J. Jung, and R. David, Generation of Murine Cardiac Pacemaker Cell Aggregates Based on ES-Cell-Programming in Combination with Myh6-Promoter-Selection. J Vis Exp, 2015(96)
DE 10 2013 114 671 A1
WO 2015/091157 A1

The present invention is illustrated in more detail by the following example.

### Example

Coupling of iSABs and Ventricular Cardiomyocytes

Induced sinoatrial bodies (“iSABs”) were prepared as described in Jung et al., (2014) and Rimmbach et al., (2015). On day 28 of differentiation, rapidly (>300 bpm) and regularly beating iSABs were picked under a light microscope. The medium of the neonatal murine cardiomyocytes seeded on the day before by means of the Primary Cardiomyocyte Isolation Kit (Pierce, USA) and grown overnight was aspirated and iSAB medium was added. 10 iSABs were seeded per well of a 24-well plate. The plates were incubated at 37° C. and 5% CO2 for 24 h in order to give the iSABs sufficient time for growth. After a medium change, the plate was searched under a light microscope for grown iSABs. With the aid of Zeiss ZENblack software, both the frequency of the cardiomyocytes and that of the iSABs was determined and checked for synchronization.

On the basis of these results, it is evident that, by maintaining iSABs in coculture with ventricular cardiomyocytes, it was possible to generate therefrom first simple conductoid constructs, in this case still without spatial separation, for reconstructing cardiac conduction. This means that it was in principle also possible to provide evidence for the feasibility of the approach for medicament testing.

1. A method for determining cardiac conduction, comprising:
(i) providing induced sinoatrial bodies (iSABs) comprising cardiac pacemaker cells;
(ii) providing working-myocardium cardiomyocytes;
(iii) measuring the cardiac conduction;
the induced sinoatrial bodies according to (i) and the working-myocardium cardiomyocytes according to (ii) being arranged in a spatially separated manner, but in conductive communication with one another.

2. The method as claimed in claim 1, comprising
comparison of the cardiac conduction measured in (iii) with a reference value, the comparison preferably comprising
(iv) administering a reference substance;
(v) measuring the cardiac conduction in the presence of the reference substance according to (iv);
(vi) comparing the values measured according to (iii) and the values measured according to (v).

3. The method as claimed in claim 2, wherein the reference substance is selected from the group of heart rate-lowering substances or from the group of heart rate-raising substances, preferably from the group consisting of isoprenaline, ZD-7288, zatebradine and ivabradine.

4. The method as claimed in claim 2, comprising
(vii) washing out the reference substance according to (iv), (v);
(viii) measuring the cardiac conduction;
(ix) optionally comparing the values measured according to (viii) and the values measured according to (iii) or according to (v); or comparing the values measured according to (viii) and the values measured according to (iii) and the values measured according to (v).

5. A method for determining the effect of substances on cardiac conduction, comprising:
(i) providing induced sinoatrial bodies (iSABs) comprising cardiac pacemaker cells;
(ii) providing working-myocardium cardiomyocytes;
(iii) optionally measuring the cardiac conduction;
(iv) administering a substance to be investigated;
(v) measuring the cardiac conduction in the presence of the substance to be investigated according to (iv);
(vi) optionally comparing the cardiac conduction measured according to (iii) and the cardiac conduction measured according to (v);
the induced sinoatrial bodies according to (i) and the working-myocardium cardiomyocytes according to (ii) being arranged in a spatially separated manner, but in conductive communication with one another.

6. A method for identifying substances which have an effect on cardiac conduction, comprising:
(i) providing induced sinoatrial bodies (iSABs) comprising pacemaker cells;
(ii) providing working-myocardium cardiomyocytes;
(iii) optionally measuring the cardiac conduction;
(iv) administering a substance to be investigated;
(v) measuring the cardiac conduction;
(vi) optionally comparing the cardiac conduction measured according to (iii) and the cardiac conduction measured according to (v); and optionally determining whether the substance to be investigated has an effect on cardiac conduction on the basis of the comparison according to (vi);
the induced sinoatrial bodies according to (i) and the working-myocardium cardiomyocytes according to (ii) being arranged in a spatially separated manner, but in conductive communication with one another.

7. The method as claimed in claim 5, comprising
(vii) washing out the substance to be investigated according to (iv)
(vii) measuring the cardiac conduction;
(viii) optionally comparing the values measured according to (vii) and the values measured according to (iii) or according to (v); or comparing the values measured according to (vii) and the values measured according to (iii) and the values measured according to (v);
(ix) optionally determining whether the substance to be investigated has an effect on cardiac conduction on the basis of the comparison according to (viii).

8. The method as claimed in claim 1, wherein the working-myocardium cardiomyocytes according to (ii) comprise atrial cells or ventricular cells or atrial and ventricular cells, preferably (ii.1) atrial and (ii.2) ventricular cells, preference being given to the induced sinoatrial bodies according to (i), the atrial cells according to (ii.1) and the ventricular cells according to (ii.2) being arranged in a spatially separated manner, but in conductive communication with one another.

9. The method as claimed in claim 1, which is carried out in vitro.

10. The method as claimed in claim 1, wherein the induced sinoatrial bodies (iSABs) comprising pacemaker cells are generated from multipotent or pluripotent stem cells, preferably from pluripotent stem cells.

11. The method as claimed in claim 1, wherein the induced sinoatrial bodies (iSABs) comprising pacemaker cells are generated from nonhuman embryonic stem cells or nonhuman induced pluripotent stem cells or human induced pluripotent stem cells or parthenogenetic stem cells or spermatogonial stem cells, preferably from nonhuman embryonic stem cells or nonhuman induced pluripotent stem cells or human induced pluripotent stem cells.

12. A device for determining a cardiac conduction, comprising:
(a) a first region 1 suitable for accommodating induced sinoatrial bodies (iSABs) comprising pacemaker cells;
(b) a second region 2 suitable for accommodating working-myocardium cardiomyocytes;
(c) a third region 3 which is suitable for allowing a conduction from the first region 1 into the second region 2.

13. The device for determining a cardiac conduction as claimed in claim 12, comprising:
(a) a first region 1 containing induced sinoatrial bodies (iSABs) comprising pacemaker cells;
(b) a second region 2 suitable for accommodating working-myocardium cardiomyocytes;
(c) a third region 3 which is suitable for allowing a conduction from the first region 1 into the second region 2.

14. The device as claimed in claim 12, wherein the second region 2 according to (b) comprises:
(b.1) a region 2.1 which is suitable for accommodating atrial cells;
(b.2) a region 2.2 which is suitable for accommodating ventricular cells;
the third region 3 according to (c) comprising:
(c.1) a region 3.1 which is suitable for allowing a conduction from the first region 1 according to (a) into the region 2.1 according to (b.1);
(c.2) a region 3.2 which is suitable for allowing a conduction from the region 2.1 according to (b.1) into the region 2.2 according to (b.2).

15. The device as claimed in claim 12, wherein the device of a material selected from the group consisting of glass, ceramic, plastic and a mixture of two or more of these materials, the plastic preferably being selected from the group consisting of polystyrene, polyethylene, polypropylene, polyethylene terephthalate and mixtures of two or more of these substances, preferably polystyrene.

16. A kit for determining a cardiac conduction, comprising
(A) a device comprising:
(a) a first region 1 suitable for accommodating induced sinoatrial bodies (iSABs);
(b) a second region 2 suitable for accommodating working-myocardium cardiomyocytes;
(c) a third region 3 which is in conductive communication with the first and the second region and is suitable for allowing a conduction from the first region 1 into the second region 2;
(B) induced sinoatrial bodies (iSABs) comprising pacemaker cells.

17. The kit as claimed in claim 16, wherein the second region 2 according to (b) comprises:
(b.1) a region 2.1 which is suitable for accommodating atrial cells;
(b.2) a region 2.2 which is suitable for accommodating ventricular cells;
the third region 3 according to (c) comprising:
(c.1) a region 3.1 which is suitable for allowing a conduction from the first region 1 according to (a) into the region 2.1 according to (b.1);
(c.2) a region 3.2 which is suitable for allowing a conduction from the region 2.1 according to (b.1) into the region 2.2 according to (b.2).

18. The kit as claimed in claim 16, wherein the device consists of a material selected from the group consisting of glass, ceramic, plastic and a mixture of two or more of these materials, the plastic preferably being selected from the group consisting of polystyrene, polyethylene, polypropylene, polyethylene terephthalate and mixtures of two or more of these substances, preferably polystyrene.

19. The use of a device as claimed in claim 12 for the in vitro evaluation of drugs or for the identification of substances which have an effect on cardiac conduction.

20. The use of a device as claimed in claim 12 for the diagnosis or prediagnosis, especially for the in vitro diagnosis or in vitro prediagnosis, of a disturbed cardiac conduction in a test subject or for the determination of the risk of a test subject developing a disturbed cardiac conduction.
